(19) United States
(12) Patent Application Publication
(10) Pub. No.: US 2024/0033209 A1
(43) Pub. Date: Feb. 1, 2024

(54) HIGH ANTIOXIDANT GRAPE CONCENTRATE AND ITS USE IN COSMETICS

(71) Applicant: Juice Beauty, Inc., Petaluma, CA (US)

(72) Inventors: Karen Behnke, Healdsburg, CA (US); Howard Luria, Healdsburg, CA (US); Julie Castel, Montpellier (FR); Angella Sprauve, San Francisco, CA (US)

(73) Assignee: Juice Beauty, Inc., Petaluma (US)

(21) Appl. No.: 18/227,890

(22) Filed: Jul. 28, 2023

Related U.S. Application Data
(60) Provisional application No. 63/393,817, filed on Jul. 29, 2022.

(51) Int. Cl. A61K 8/9789; A61Q 19/08

(52) U.S. Cl. CPC A61K 8/9789; A61Q 19/08; A61K 2800/522; A61K 2800/51; A61K 2800/85

(57) ABSTRACT

Disclosed are topical cosmetic compositions comprising a fermented grape concentrate having high antioxidant levels, and a cosmetically acceptable ingredient. Also disclosed are methods of using the topical cosmetic compositions to improve a condition or appearance of skin.

### BACKGROUND OF THE INVENTION

There are hundreds of skin care products on the market today designed to provide a variety of benefits and care for hands, feet, body, eyes, face, etc. Skin care products touting antioxidant properties represent an important sector of so-called cosmetic products because antioxidants do, in fact, provide skin benefit by scavenging free-radicals. Free radicals are highly reactive molecules with unpaired electrons that can directly damage various cellular structural membranes, lipids, proteins, and DNA (see, Bigdan, et al., “Antioxidants used in skin care formulations,” Skin Therapy Lett., 2008, 13(7):5-9). Free radicals bring an oxidative stress to the skin that results in accelerated apparition of sign of aging. Thus, it would be advantageous and beneficial to have topical skin care products that contain high levels of antioxidants that can be used to slow down or prevent the signs of aging, such as wrinkles, fine lines, lack of elasticity or firmness.

### BRIEF SUMMARY OF THE INVENTION

Provided are topical skin care products containing high levels of phenolic compounds and, in turn, high levels of antioxidants that can advantageously be used to slow down and/or prevent the signs of aging, i.e., slow down and/or prevent wrinkles, fine lines, lack of elasticity or firmness, etc. The present invention provides a fermented grape (Vitis vinifera) concentrate having high total phenolic content and high antioxidant levels that can advantageously and beneficially be used in topical cosmetic products to neutralize skin-damaging free radicals, to hydrate the skin, and to improve the appearance of fine lines, wrinkles, and discoloration. The fermented grape concentrate is derived from Sagrantino grapes, which have been cultivated to be a great source of antioxidants due to the high amount of phenolic compounds found in both the skins and the seeds of Sagrantino grapes. It has been surprisingly discovered that the antioxidant levels of the fermented grape concentrate made from Sagrantino grapes are orders of magnitude higher than the antioxidant levels found in most grape juices (e.g., conventional and organic grape juice) used in topical skin care products currently on the market. The fermented grape concentrate provided by the present invention contains both high levels of phenolic compounds and high levels of antioxidants, giving the fermented grape concentrate, both on its own and when used in topical cosmetic compositions/products, the ability to neutralize skin-damaging free radicals, to hydrate the skin, and to improve the appearance of fine lines, wrinkles, and discoloration.

In one aspect, the present invention provides a fermented grape concentrate, wherein the grape concentrate has one or more of the following characteristics: (i) an Oxygen Radical Absorbance Capacity (ORAC) value ranging from about 10 μM TE/g to about 60 μM TE/g, (ii) a total phenolic content ranging from about 1000 mg/L to about 4500 mg/L; (iii) a sugar content of 3° Bx or less; and (iv) an alcohol by volume (ABV) of between about 0% and about 6%.

In another aspect, the present invention provides a topical cosmetic composition, the cosmetic composition comprising: a fermented grape (Vitis vinifera) concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and a cosmetically acceptable ingredient; wherein the pH of the cosmetic composition ranges from about 4.0 to about 6.5. In one embodiment, the fermented grape concentrate has one or both of the following characteristics: (i) a sugar content of 3° Bx or less; and/or (ii) an alcohol by volume (ABV) of between about 0% and about 6%.

In one embodiment, the fermented grape concentrate has an ORAC value ranging from 15 μM TE/g to about 50 μM TE/g. In another embodiment, the fermented grape concentrate has an ORAC value ranging from about 20 μM TE/g to about 40 μM TE/g. In yet another embodiment, the fermented grape concentrate has a total phenolic content ranging from about 1500 mg/L to about 3600 mg/L. In one embodiment, the pH of the cosmetic composition ranges from about 4.8 to about 5.8. In another embodiment, the pH of the cosmetic composition is greater than 5.0. In preferred embodiments, the fermented grape concentrate used in the topical cosmetic compositions of the present invention is a Sagrantino grape concentrate, i.e., the grape used to make the fermented grape concentrate is a Sagrantino grape.

Cosmetic composition containing the fermented grape concentrate can include, but are not limited to the following: moisturizers, masks, body care products, eye care products, primers, foundations, concealers, cream blushes, lipsticks, lip balms, mascaras, shampoos, hair conditioners and cream rinses, shower gels, sunscreens, facial cleanser, etc. In one embodiment, the cosmetic composition is a moisturizer, e.g., a lotion, a cream, a gel, a serum, etc. In one embodiment, the cosmetic composition is a serum and, in particular, a stem cellular, supergrape, youth renew serum that contains both the fermented grape concentrate disclosed herein as well as other cosmetically acceptable ingredients.

In one aspect, the cosmetically acceptable ingredient includes, but is not limited to, one or more of the following: an active agent, a solvent, a humectant, a rheology modifying agent, an emollient, an emulsifying agent, a preservative, a chelating agent, a buffering agent, a colorant, a fragrance, a UV filtering agent, and combinations thereof. In another aspect, the cosmetically acceptable ingredient includes a combination of an active agent, a solvent, a humectant, a rheology modifying agent, and a preservative, and can optionally further comprise one or more cosmetically acceptable ingredients including, but not limited to, the following: an emollient, an emulsifying agent, a buffering agent, a colorant, a fragrance, and a UV filtering agent. In one embodiment, the cosmetically acceptable ingredient includes an active agent, a solvent, a humectant, a rheology modifying agent, an emollient, an emulsifying agent, a preservative, a buffering agent (or a pH adjusting agent), and a fragrance (or scent).

In another aspect, the present invention provides a method of improving a condition or appearance of skin, the method comprising applying to the skin an effective amount of a topical cosmetic composition, the composition comprising: the cosmetic composition comprising: a fermented grape (Vitis vinifera) concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and a cosmetically acceptable ingredient; wherein the pH of the cosmetic composition ranges from about 4.0 to about 6.5. In one embodiment, the fermented grape concentrate has one or both of the following characteristics: (i) a sugar content of 3° Bx or less; and/or (ii) an alcohol by volume (ABV) of between about 0% and about 6%. In one embodiment, the topical composition is a moisturizer, such as a lotion, a cream, a gel, or a serum.

The methods and cosmetic compositions of the present invention can be used, for example, to hydrate the skin, to increase moisture level in the skin, to increase skin radiance, to inhibit melanogenesis and/or tyrosinase, to inhibit hyperpigmentation, to increase collagen production in the skin, etc. In one embodiment, the methods and compositions of the present invention can be used, for example, to improve a skin condition, wherein the skin conditions is selected from skin radiance, skin tone clarity, skin brightness, skin tone evenness, and combinations thereof. In one embodiment, the methods and compositions of the present invention can be used to reduce a skin condition, wherein the skin condition is photo damage, loss of facial firmness, fine lines, deep lines, wrinkles, skin dullness, skin sagging, appearance of age spots on skin, or combinations thereof. In view of the high levels of phenolic compounds and antioxidants, it should be appreciated that the topical cosmetic compositions of the present invention can be used in other methods to neutralize skin-damaging free radicals, to hydrate the skin, and to improve the appearance of fine lines, wrinkles, and discoloration.

In another aspect, the present invention provides a method for preparing a topical cosmetic composition, the method comprising: (a) combining (i) a fermented grape concentrate, wherein the grape concentrate has an Oxygen Radical Absorbance Capacity (ORAC) value ranging from about 10 μM TE/g to about 50 μM TE/g, and a total phenolic content ranging from about 1000 mg/L to about 4500 mg/L, and (ii) a cosmetically acceptable ingredient to form a product; and (b) adjusting the pH of the product to a pH of about 4.0 to about 6.5, thereby preparing the topical cosmetic composition. In one embodiment of this method, the fermented grape concentrate has one or both of the following characteristics: (i) a sugar content of 3° Bx or less; and/or (ii) an alcohol by volume (ABV) of between about 0% and about 6%.

In yet another aspect, the present invention provides a composition for use in a topical cosmetic product, the composition comprising a fermented grape concentrate, wherein the grape concentrate has an Oxygen Radical Absorbance Capacity (ORAC) value ranging from about 10 M TE/g to about 60 μM TE/g, and a total phenolic content ranging from about 1000 mg/L to about 4500 mg/L, and wherein the grape concentrate is stable at a pH of about 4.0 to about 6.5. In one embodiment of this method, the fermented grape concentrate has one or both of the following characteristics: (i) a sugar content of 3° Bx or less; and/or (ii) an alcohol by volume (ABV) of between about 0% and about 6%.

### DETAILED DESCRIPTION OF THE INVENTION

### A. Introduction

The present invention provides a fermented grape (Vitis vinifera) concentrate having high total phenolic content and high antioxidant levels that can advantageously and beneficially be used in topical cosmetic products to neutralize skin-damaging free radicals, to hydrate the skin, and to improve the appearance of fine lines, wrinkles, and discoloration. The fermented grape concentrate is derived from Sagrantino grapes, and it has surprisingly been discovered that the antioxidant levels of the fermented grape concentrate made from Sagrantino grapes are orders of magnitude higher than the antioxidant levels found in most grape juices (e.g., Fruit Smart grape juice) used in topical skin care products currently on the market. The fermented grape concentrate of the present invention is a great source of antioxidants due to the high amount of phenolic compounds found in both the skins and the seeds of the Sagrantino grapes from which it is derived. In view of its high levels of antioxidants, the fermented grape concentrate can be used to make topical cosmetic compositions that also have high levels of antioxidants and that can be used, for example, to neutralize skin-damaging free radicals. The cosmetic compositions of the present invention are antioxidant compositions that can beneficially be used, for example, to hydrate the skin, and to improve the appearance of fine lines, wrinkles, and discoloration. The antioxidant cosmetic compositions/products of the present invention include, but not limited to, moisturizers (e.g., lotions, creams, gels, serums), masks, body care products, eye care products, primers, foundations, concealers, cream blushes, lipsticks, lip balms, mascaras, shampoos, hair conditioners and cream rinses, shower gels, sunscreens, facial cleanser, etc., all of which can be used to combat the signs of aging.

### B. Fermented Grape Concentrate

In one aspect, the present invention provides a fermented grape concentrate, wherein the grape concentrate has one or more of the following characteristics: (i) an Oxygen Radical Absorbance Capacity (ORAC) value ranging from about 10 μM TE/g to about 60 μM TE/g, (ii) a total phenolic content ranging from about 1000 mg/L to about 4500 mg/L; (iii) a sugar content of 3° Bx or less; and (iv) an alcohol by volume (ABV) of between about 0% and about 6%. In one embodiment, the fermented grate concentrate has two or more of the above characteristics (i)-(iv). In other embodiments, the fermented grate concentrate has three or more of the above characteristics (i)-(iv). In yet another embodiment, the fermented grape concentrate has all four of the above characteristics (i)-(iv). In a preferred embodiment, the fermented grape concentrate is a Sagrantino grape concentrate, i.e., the grapes used to make the fermented grape concentrate are Sagrantino grapes. Sagrantino grapes are a rich source of phenolic compounds relative to other grapes, such as green grapes and other red grapes, and other fruits, and the phenolic compounds found in Sagrantino grapes have strong antioxidant activity, making the fermented grape concentrate derived from Sagrantino grapes a powerful and strong inhibitor of free radical-mediated reactions. Examples of phenolic compounds found in Sagrantino grapes and, in turn, in the fermented grape concentrate of the invention include, but are not limited to, flavonols, dihydroflavonols, flavanol monomers (e.g., catechins), flavanol polymers (e.g., proanthocyanidins) anthocyanins, quercetins, stilbenoids, phenolic acids, and derivatives thereof. In other embodiments, the fermented grape concentrate is a Cabernet or a Falanghina grape concentrate.

Importantly, the terms“about” and“around,” as used herein to modify a numerical value, indicate a close range surrounding that explicit value. If “X” were the value, “about X” or “around X” would indicate a value from 0.9X to 1.1X, and more preferably, a value from 0.95X to 1.05X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

In one embodiment, the fermented grape concentrate has an ORAC value ranging from 15.0 μM TE/g to about 50.0 μM TE/g. In another embodiment, the fermented grape concentrate has an ORAC value ranging from about 20.0 μM TE/g to about 40.0 μM TE/g. In another embodiment, the fermented grape concentrate has an ORAC value ranging from about 22.0 μM TE/g to about 38.0 μM TE/g. In another embodiment, the fermented grape concentrate has an ORAC value ranging from about 25.0 μM TE/g to about 35.0 μM TE/g. In yet another embodiment, the fermented grate concentrate has an ORAC value of 20.0 μM TE/g, 21.0 μM TE/g, 22.0 μM TE/g, 23.0 μM TE/g, 24.0 μM TE/g, 25.0 μM TE/g, 26.0 μM TE/g, 27.0 μM TE/g, 28.0 μM TE/g, 29.0 μM TE/g, 30.0 μM TE/g, 31.0 μM TE/g, 32.0 μM TE/g, 33.0 μM TE/g, 34.0 μM TE/g, 35.0 μM TE/g, 36.0 μM TE/g, 37.0 μM TE/g, 38.0 μM TE/g, 39.0 μM TE/g, 40.0 μM TE/g, 41.0 μM TE/g, 42.0 μM TE/g, 43.0 μM TE/g, 44.0 μM TE/g, 45.0 μM TE/g, 46.0 μM TE/g, 47.0 μM TE/g, 48.0 μM TE/g, 49.0 μM TE/g, 50.0 μM TE/g, 51.0 μM TE/g, 52.0 μM TE/g, 53.0 μM TE/g, 54.0 μM TE/g, 55.0 μM TE/g, 56.0 μM TE/g, 57.0 μM TE/g, 58.0 μM TE/g, 59.0 μM TE/g, 60.0 μM TE/g. Importantly, the ORAC value of the fermented grape concentrate can include a range of any of the foregoing ORAC values, e.g., the fermented grape concentrate can have an ORAC value that ranges from about 25.0 μM TE/g to about 33.0 μM TE/g, or from about 30.0 μM TE/g to about 42.0 μM TE/g, etc.

As will be appreciated by those of ordinary skill in the art, the ORAC unit (Oxygen Radical Absorbance Capacity), ORAC value, or ORAC score is a method initially developed by scientists at the National Institute of Health and Aging (NIH) that can be used to measures the antioxidant capacity of the fermented grape concentrate disclosed herein, either on its own, or in combination with any (one or more) cosmetically acceptable ingredient(s) or in any of the topical cosmetic compositions disclosed herein. Antioxidant activity indicates a capability to reduce oxidizing agents (oxidants). This assay quantifies the degree and length of time it takes to inhibit the action of an oxidizing agent, such as oxygen radicals, which are known to cause damage to cells (e.g., skin cells). The ORAC value of any one of the fermented grape concentrate or any of the cosmetically acceptable ingredients, combination of ingredients, or compositions having any of the combinations disclosed herein can be determined by methods known to and used by those of ordinary skill in the art (see, e.g., U.S. Publication Nos. 2004/0109905 and 2005/0163880; Ningali et al., “Validation of the Oxygen Radical Absorbance Capacity (ORAC) Parameters as a New Index of Quality and Stability of Virgin Olive Oil,” JAOCS, 2002, vol. 79, no. 10, 977-982; and commercially available kits such as Zen-Bio ORAC Anti-oxidant Assay kit (#AOX-2)). The Zen-Bio ORAC Antioxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

In one embodiment, the fermented grape concentrate has a total phenolic content ranging from about 1500 mg/L to about 3800 mg/L. In another embodiment, the fermented grape concentrate has a total phenolic content ranging from 1800 mg/L to about 3600 mg/L. In yet another embodiment, the fermented grape concentrate has a total phenolic content ranging from 1800 mg/L to 2200 mg/L. In another embodiment, the fermented grape concentrate has a total phenolic content ranging from 3000 mg/L to about 3800 mg/L. In yet another embodiment, the fermented grate concentrate has a total phenolic content of 1000 mg/L, 1100 mg/L, 1200 mg/L, 1300 mg/L. 1400 mg/L, 1500 mg/L, 1600 mg/L, 1700 mg/L, 1800 mg/L, 1900 mg/L, 2000 mg/L, 2100 mg/L, 2200 mg/L, 2300 mg/L. 2400 mg/L, 2500 mg/L, 2600 mg/L, 2700 mg/L, 2800 mg/L, 2900 mg/L, 3000 mg/L, 3100 mg/L, 3200 mg/L, 3300 mg/L. 3400 mg/L, 3500 mg/L, 3600 mg/L, 3700 mg/L, 3800 mg/L, 3900 mg/L, 4000 mg/L, 4100 mg/L, 4200 mg/L, 4300 mg/L. 4400 mg/L, or 4500 mg/L. Importantly, the total phenolic content of the fermented grape concentrate can include a range of the foregoing values, e.g., the fermented grape concentrate can have a total phenolic content ranging from about 1800 mg/L to about 2300 mg/L, or from 1800 mg/L to about 3200 mg/L.

The total phenolic content of the fermented grape concentrate can be determined via a spectrophotometric method including, but not limited to, the Folin-Ciocalteau (F-C) Phenolic Content Quantification Assay. In a preferred embodiment, total phenolic content is evaluated using the Folin-Ciocalteau assay, an assay which is known to and used by those of ordinary skill in the art to measure and assess phenolic content. For this colorimetric assay, a standard curve is prepared using increasing concentrations of a reference molecule. In preferred embodiments, the reference polyphenol used in this assay is gallic acid. The determination of the total phenolic content in the fermented grape concentrate is then obtained using the same protocol as that carried out for the standard range. For more information on the Folin-Ciocalteau assay, see, e.g., Waterhouse, A. L., Determination of Total Phenolics, in Current Protocols in Food Analytical Chemistry, I1.1.1-I1.1.8, Wrolstad, RE., Wiley, 2001, or Singleton et al., “Analysis of total phenols and other oxidation substrates and antioxidants by means of Folin-Ciocalteu Reagent,” Methods in Enzymology, 1999, 299, 152-178, or Sanchez-Rangel et al., “The Folin-Ciocalteu assay revisited: improvement of its specificity for total phenolic content determination,” Anal. Methods, 2013, 5, 5990-5999.

During the processing of the grapes, i.e., the Sagrantino grapes, the sugar content is reduced using techniques known to those of skill in the art. In one embodiment, the sugar content is reduced or removed through alcoholic (ethanolic) fermentation. It should be appreciated that any method or technique known to those having ordinary skill in the art may be used to reduce the sugar content. In one embodiment, the fermented grape concentrate has a sugar content of 3 degrees brix, i.e., 3° Bx, or less. In another embodiment, the femented grape concentrate has a sugar content of 2° Bx or less. In yet another embodiment, the femented grape concentrate has a sugar content of 1° Bx or less. In other embodiments, the femented grape concentrate has a sugar content ranging from about 0° Bx to about 3° Bx, more preferably, from about 0.01° Bx to about 2.5° Bx, and, even more preferably, from about 0.05° Bx to about 1° Bx. In other embodiments, the femented grape concentrate has a sugar content of 0° Bx to about 3° Bx, more preferably, from about 0.01° Bx to about 2.5° Bx, and, even more preferably, from about 0.05° Bx to about 1° Bx. As is known to those of ordinary skill in the art, each degree brix (° 1) is typically equivalent to 1% sugar concentration (1 gram of sugar (e.g., sucrose) in 100 grams of solution) when measured at 20° C. Brix measurements are typically made at the end of the fermentation and extraction process, which can range anywhere from 14 to 21 days, and can be conducted using densiometery or using “Brix Meter” or “Brix Refractometer.” Brix refractometers are a common instrument used to measure the sugar content of a solution. Increasing sugar content leads to an increase in the refractive index. By passing light through a sample and measuring the refraction, the amount that the light bends, the sugar content can be measured.

During the processing of the grapes, i.e., the Sagrantino grapes, the alcohol by volume is reduced or removed using techniques known to those of skill in the art. In one embodiment, the alcohol, e.g., ethanol, is removed through a spinning cone column, or through a “clean” reverse osmosis membrane process. Those of ordinary skill will appreciate that other techniques and methodologies can also be used to reduce the alcohol by volume of the fermented grape concentrate. In one embodiment, the fermented grape concentrate has an alcohol by volume (ABV) of between about 0% and about 6%. In another embodiment, the fermented grape concentrate has an ABV of between 0.01% and about 4%. In another embodiment, the fermented grape concentrate has an ABV of between 0.50% and about 4%. In another embodiment, the fermented grape concentrate has an ABV of between 1.0% and about 2.5%. It should be appreciated that “ABV” is a standard measure of how much alcohol is contained in a given volume of an alcoholic solution. During fermentation, the yeast consumes the sugars of the crushed raw grape pulp to produce alcohol, i.e., ethanol. The density of sugar in the water is greater than the density of alcohol in the water. In some embodiments, the ABV of the fermented grape concentrate was measured by gas chromatography at the end of the fermentation and extraction process, which can range anywhere from 14 to 21 days. In other embodiments, a hydrometer is used to measure a change in the specific gravity (SG) of the solution before and after the fermentation or during two time periods during the fermentation process. The volume of the alcohol in a grape-type/wine-type solution can then be estimated via numerous methods, where one such method includes the following: ABV=136×(Starting SG-Final SG or Starting SG₁−SG₂, wherein SG₁ and SG₂, are the SG at time period #1 and the SG at time period #2). It should be appreciated that any method known to those having ordinary skill in the art may be used to estimate the volume of the alcohol in the solution or the resulting fermented grape concentrate.

### C. Cosmetic Compositions

Sagrantino grapes are a rich source of phenolic compounds, and the phenolic compounds found in Sagrantino grapes have strong antioxidant activity, making the fermented grape concentrate derived from Sagrantino grapes a powerful and strong inhibitor of free radical-mediated reactions. Because it is rich in phenolic compounds and has antioxidant activity, the fermented grape concentrate can advantageous be added to a cosmetic composition to impart antioxidant properties to that cosmetic composition. The fermented grape concentrate disclosed herein can be added to any cosmetic composition or product that would benefit from antioxidant properties. Such cosmetic compositions and products that would benefit from the addition of the antioxidant properties of the fermented grape concentrate include, but are not limited to, the following: moisturizers (e.g., lotions, creams, gels, or serums), masks (e.g., face, body, hand, or eye masks), body care products, eye care products, primers, foundations, concealers, cream blushes, lipsticks, lip balms, mascaras, shampoos, hair conditioners/cream rinses, shower gels, facial cleansers, sun screens, etc.

The cosmetic compositions of the present invention are typically topical cosmetic compositions, i.e., cosmetic compositions for topical application. “Topical application” means to apply or spread a composition onto the surface of lips or keratinous tissue. “Topical cosmetic composition” or “topical skin composition” includes compositions suitable for topical application on skin and/or keratinous tissue. Such compositions are typically dermatologically-acceptable in that they do not have undue toxicity, incompatibility, instability, allergic response, and the like, when applied to skin and/or keratinous tissue. Topical skin care compositions of the present invention can have a selected viscosity to avoid significant dripping or pooling after application to skin and/or keratinous tissue.

In one aspect, the present invention provides a topical cosmetic composition, the cosmetic composition comprising: a fermented grape (Vitis vinifera) concentrate, wherein the grape concentrate has an Oxygen Radical Absorbance Capacity (ORAC) value ranging from about 10 μM TE/g to about 60 μM TE/g, and a total phenolic content (TPC) ranging from about 1000 mg/L to about 4500 mg/L; and a cosmetically acceptable ingredient; wherein the pH of the cosmetic composition ranges from about 4.0 to about 6.5.

In another aspect, the present invention provides a topical cosmetic composition, the cosmetic composition comprising: a fermented grape concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and a cosmetically acceptable ingredient, wherein the cosmetically acceptable ingredient comprises an active agent; a solvent, a humectant, a rheology modifying agent, and a preservative; wherein the pH of the cosmetic composition ranges from about 4.0 to about 6.0.

In yet another aspect, the present invention provides a topical cosmetic composition, the cosmetic composition comprising: a fermented grape concentrate, wherein the grape concentrate has an Oxygen Radical Absorbance Capacity (ORAC) value ranging from about 10 M TE/g to about 60 μM TE/g, and a total phenolic content ranging from about 1000 mg/L to about 4500 mg/L; and a cosmetically acceptable ingredient, wherein the cosmetically acceptable ingredient comprises an active agent, a solvent, a humectant, an emulsifying agent, an emollient, a rheology modifying agent, a buffering agent, a preservative, and a fragrance; wherein the pH of the cosmetic composition ranges from about 4.0 to about 6.5.

Additional properties and characteristics of the fermented grape concentrate used in the cosmetic compositions disclosed herein are described in Section A.—Fermented Grape Concentrated.

The pH of the cosmetic composition will generally range from about 4.0 to about 6.5. In one embodiment, the pH of the cosmetic composition ranges from about 4.8 to about 5.8. In another embodiment, the pH is greater than 5.0. In most embodiments, the cosmetic composition will have a pH of 4.0, 4.1, 4.2, 4.3, 4.4, 4.5, 4.6, 4.7, 4.8, 4.9, 50. 5.1, 5.2, 5.3, 5.4, 5.5, 5.6, 5.7, 5.8, 5.9, 6.0, 6.1, 6.2, 6.3, 6.4, or 6.5. Those of ordinary skill will appreciate that the pH of the cosmetic composition will be adjusted to be appropriate for the intended use of the cosmetic composition. Those of ordinary skill will also appreciate that the pH of the cosmetic composition can readily be adjusted using a buffer or pH adjusting agent, such as an acid or base.

The cosmetically acceptable ingredients are selected so that the cosmetic compositions of the present invention are cosmetically elegant or can have pleasant tactile properties. “Cosmetically elegant,” and/or “pleasant tactile properties” generally describes a composition that has particular tactile properties which feel pleasant on the skin (e.g., compositions that are not too watery or greasy, compositions that have a silky texture, compositions that are non-tacky or sticky, etc.). Cosmetically elegant can also relate to the creaminess or lubricity properties of the composition or to the moisture retaining properties of the composition.

The cosmetically acceptable ingredient used in the cosmetic compositions disclosed herein includes, but is not limited to, one or more of the following: an active agent, a solvent, a humectant, a rheology modifying agent, an emollient, an emulsifying agent, a preservative, a chelating agent, a buffering agent, a colorant, a fragrance, a UV filtering agent, and combinations thereof. In another aspect, the cosmetically acceptable ingredient includes a combination of an active agent, a solvent, a humectant, a rheology modifying agent, and a preservative, and can optionally further comprise one or more cosmetically acceptable ingredients including, but not limited to, the following: an emollient, an emulsifying agent, a buffering agent, a colorant, a fragrance, and a UV filtering agent. In one embodiment, the cosmetically acceptable ingredient includes an active agent, a solvent, a humectant, a rheology modifying agent, an emollient, an emulsifying agent, a preservative, a buffering agent (or a pH adjusting agent), and a fragrance (or scent). Those of ordinary skill will appreciate that the cosmetically acceptable ingredients used in the cosmetic products disclosed herein will depend on the cosmetic product itself and the intended use of the cosmetic product. For instance, the cosmetically acceptable ingredients used in a serum, for example, may be the same as, different from, or share some commonality with the cosmetically acceptable ingredients used in a lip balm or a shampoo. Non-limiting examples of the cosmetically acceptable ingredients are described in the following subsections.

1. Active Agents

When used in a cosmetic composition, the fermented grape concentrate to impart antioxidant properties to that cosmetic composition as a result of the phenolic compounds present in the concentrate. In addition to the fermented grate concentrate, the cosmetic compositions can also include, if desirable, one or more additional active agents. Non-limiting examples of active agents that can be used with the cosmetic compositions of the present invention include vitamins, vitamin derivatives, amino acids, amino acid derivatives, peptides, peptide derivatives, proteins, protein derivatives, polysaccharides and polysaccharide derivatives, plant extracts, alpha-, beta-, and poly-hydroxy acids (AHA), urea; uric acid derivatives, and combinations thereof. Those of ordinary skill will appreciate that other active agents can be included the cosmetic compositions/products disclosed herein and will depend on the cosmetic product itself and the intended use of the cosmetic product.

Examples of vitamins and vitamin derivatives suitable for use in the cosmetic compositions include, but are not limited to, the following: tocopherol (Vitamin E), ascorbic acid (Vitamin C), panthenol, retinol (Vitamin A), niacinamide, and combinations thereof

Examples of amino acids and amino acid derivatives suitable for use in the cosmetic compositions include, but are not limited to, the following: arginine, histidine, methionine, lysine, proline, leucine, glycine, and combinations thereof.

Examples of peptides and peptide derivatives suitable for use in the cosmetic compositions include, but are not limited to, the following: acetyl hexapeptide-8, dipeptide-2, hexapeptide-11, hydrolyzed wheat peptides, oligopeptide-1.

Examples of peptides and peptide derivatives suitable for use in the cosmetic compositions include, but are not limited to, the following: collagen, elastine, keratin, and combinations thereof.

Examples of polysaccharides suitable for use in the cosmetic compositions include, but are not limited to, hyaluronic acid.

Examples of plant extracts suitable for use in the cosmetic compositions include, but are not limited to, the following: Opuntia ficus-indica stem extract, Malus domestica fruit cell culture extract, and Vitis vinifera (grape) extract, and combinations thereof.

Examples of hydroxy acids suitable for use in the cosmetic compositions include, but are not limited to, the following: alpha-hydroxy acids, beta-hydroxy acids, poly-hydroxy acids, and combinations thereof. Suitable hydroxy acids include, but are not limited to, lactic acid, glycolic acid, malic acid, tartaric acid, salicylic acid, gluconolactone, and combinations thereof.

An example of a uric acid derivative suitable for use in the cosmetic compositions includes, but is not limited to, allantoin. Urea can also be used as an active agent in the cosmetic compositions disclosed herein.

Those of ordinary skill will appreciate that one or more active agents can be included in the cosmetic compositions disclosed herein. For instance, the cosmetic compositions can include one, two, three, four, five, six, seven, eight, nine, ten, or more active agents. In one embodiment, the active agent comprises Opuntia ficus-indica stem extract, Malus domestica fruit cell culture extract, and Vitis vinifera (grape) extract, tocopherol, panthenol, hyaluronic acid, ascorbyl glucoside, and arginine.

2. Solvents

In certain aspects, the cosmetic compositions comprise a solvent in addition to the fermented grape concentrate. Non-limiting examples of solvents include, but are not limited to, water, a plant juice, a plant water, and combinations thereof.

Examples of plant juices suitable for use in the cosmetic compositions include, but are not limited to, the following: the plant juice is selected from the group consisting of the following: Aloe arborescens leaf juice, Aloe barbadensis leaf juice, Ananas sativus (pineapple) fruit juice, Betula alba (birch) juice, Borojoa patinoi fruit juice, Bulbine frutescens leaf juice, Citrus aurantium dulcis (Orange) fruit extract, Citrus medica limonum (Lemon) juice, Cocos nucifera (Coconut) fruit juice, Cucumis sativus (Cucumber) juice, Empetrum Nigrum (black Crowberry) Fruit Juice, Fragaria chiloensis (Strawberry) fruit juice, Hippophae rhamnoides fruit juice, Hordeum vulgare leaf juice, Musa paradisiaca fruit juice, Polymnia sonchifolia root juice, Prunus persica (Peach) juice, Pyrus malus (Apple) juice, Rubus chamaemorus (Cloudberry) fruit juice extract, Sorghum bicolor stalk juice, Vaccinium myrtillus (Bilberry) fruit juice, Vitis vinifera (Grape) juice, and combinations thereof. Other plant juice known to those of skill in the art can also be used in the cosmetic compositions disclosed herein.

Examples of plant waters suitable for use in the cosmetic compositions include, but are not limited to, the following: the plant juice is selected from the group consisting of the following: Actinidia Chinensis (Kiwi) fruit water, Ananas sativus (Pineapple) fruit water, Anthemis nobilis flower water, Citrullus lanatus (Watermelon) fruit extract, Citrullus vulgaris (Watermelon) fruit extract, Citrus aurantium dulcis (Orange) fruit water, Citrus grandis (Grapefruit) fruit water, Citrus paradisi (Grapefruit) fruit water, Citrus medica limonum (Lemon) fruit water, Ficus carica (Fig) fruit water, Fragaria ananassa (Strawberry) fruit water, Gossypium herbaceum (Cotton) fruit water, Malus domestica (apple) fruit water, Mentha piperita leaf water, Olea europaea (Olive) fruit water, Opuntia ficus-indica (prickly pear) water, Prunus armeniaca (plum) fruit water, Prunus persica (plum) fruit water, Prunus armeniaca (Apricot) fruit water, Prunus cerasus (Cherry) fruit water, Prunus persica (Peach) fruit water, Rubus fruticosus fruit (blackberry) water, Rubus idaeus (Raspberry) fruit water, Vaccinium macrocarpon (Cranberry) fruit water, Vaccinium myrtillus (bilberry) fruit water, Vanilla planifolia fruit water, Vitis vinfera (Grape) fruit water, and combinations thereof. Other plant juice known to those of skill in the art can also be used in the cosmetic compositions disclosed herein.

Those of ordinary skill will appreciate that one or more solvents can be included in the cosmetic compositions disclosed herein. For instance, the cosmetic compositions can include one, two, three, four, five, six, seven, eight, nine, ten, or more solvents, i.e., plant juices, plant waters, water, and combinations thereof. In one embodiment, the active agent comprises Aloe barbadensis leaf juice and Pyrus malus (apple) juice. In preferred embodiments, about 80%, preferably, about 85%, more preferably, about 90%, even more preferably, about 95%, and even more preferably, about 100% of the solvent is made up of plant juice(s) and/or plant water(s).

3. Humectants

Humectants include substances that promote retention of water or moisture. Non-limiting examples of humectants that can be used with the cosmetic compositions disclosed herein include, but are not limited to, the following: a glycol, a glycerin, and combinations thereof. Suitable glycols include, but are not limited to, butylene glycol, pentylene glycol, propanediol, polyethylene glycol (PPG), and combinations thereof. Glycerin and diglycerin can also be used as humectants in the cosmetic compositions disclosed herein. In one embodiment, the humectant is a combination of pentylene glycol and glycerin.

4. Rheology-Modifying Agents

Rheology-modifying agents, which are also known as thickening agents (e.g., thickeners or gelling agents), include substances that can increase the viscosity of a cosmetic composition. Typically, the rheology-modifying agent include those that can increase the viscosity of a cosmetic composition substantially modifying the efficacy of the active agent(s) within the cosmetic composition. Rheology-modifying agents can also increase the stability of the cosmetic compositions disclosed herein. Non-limiting examples of rheology-modifying agents include, but are not limited to the following: gums, carrageenans, carrageenan derivatives, carbomers, carbomer derivatives, acrylic acids, acrylic acid derivative, cellulose, cellulose derivatives, silica, silica derivative, clay, dextrin, dextran derivatives, copolymers (e.g., acrylate co-polymers and VP/VA co-polymers), and combinations thereof.

In one embodiment, the rheology-modifying agent in a gum. Examples of gums suitable for use as a rheology-modifying agent include, but are not limited to, the following: xanthan gum, algin, algin derivatives, sclerotium gum, dehydroxanthan gum, Ceratonia siliqua gum, Caesalpinia espinosa gum, cellulose gum, and combinations thereof. In another embodiment, the rheology-modifying agent can be carrageenan or a carrageenan derivative including, but not limited, to the following: chrondrus crispus powder, sodium carrageenan, and combinations thereof. In another embodiment, the rheology-modifying agent is a carbomer or a carbomer derivative including, but not limited to sodium carbomer. In another embodiment, the rheology-modifying agent can be acrylic acid or an acrylic acid derivative. Examples of suitable acrylic acid derivatives include, but are not limited to, the following: acrylates copolymer, sodium acrylates copolymer, acrylates/C10-30 alkyl acrylates cross-polymer, polyacrylate cross-polymer-6, polyquaternium-37, sodium polyacrylate, and combinations thereof. In other embodiments, the rheology-modifying agent can be a cellulose or a cellulose derivative including, but not limited to, cellulose, cellulose acetate, cellulose gum, ethyl cellulose, and combinations thereof. The rheology-modifying agent can also be a destrin or dextrin derivative. In other embodiments, the rheology-modifying agent can be a clay or a silica and derivatives thereof. Examples of suitable clays include, but are not limited to, hectorite, bentonite, and combinations thereof. Examples of suitable silica and silica derivatives include, but are not limited to, calcium silicate, silica, magnesium silicate, and combination thereof.

Those of ordinary skill will appreciate that one or more rheology-modifying agents can be included in the cosmetic compositions disclosed herein, and that the rheology-modifying agent can be a mixture or combination of the different classes or types of rheology-modifying agents. In one embodiment, the rheology-modifying agent comprises algin and xanthan gum.

5. Emollients

In certain aspects of the invention, the cosmetic compositions do not include an emollient. In other aspects, however, the cosmetic compositions can include one or more emollients. Emollients are typically substances that prevents water loss and have a softening and soothing effect on skin. Non-limiting examples of emollients suitable for use in the cosmetic compositions include, but are not limited to, triglycerides, oils, butters, waxes, silicones, mineral oil, light mineral oil, paraffin hydrocarbons, fatty alcohols, fatty acids, esters of a fatty acid with an alcohol, ceramides, phospholipids, and combinations thereof.

Those of ordinary skill will appreciate that one or more emollients can be included in the cosmetic compositions disclosed herein. In one embodiment, the emollient comprises Simmondsia chinensis (Jojoba) seed oil, Borago officinalis seed oil, Rosa canina fruit oil, Linum usitatissimum (Linseed) seed oil, Butyrospermum parkii (Shea) butter, and hydrolyzed jojoba esters.

6. Emulsifying Agents

In certain aspects of the invention, the cosmetic compositions do not include an emulsifying agent, i.e., emulsifier. In other aspects, however, the cosmetic compositions can include one or more emulsifying agents. Emulsifiers typically reduce the interfacial tension between phases and improve the composition and stability of an emulsion. The emulsifying agents used in the cosmetic compositions can be anionic emulsifiers, cationic emulsifiers, non-ionic emulsifiers, amphoteric or zwitterionic emulsifiers, and combinations thereof.

Examples of anionic emulsifiers include, but are not limited to, the following: fatty alcohol sulphates (e.g., cetearyl sulfate), isethionates (e.g., sodium cocoyl isethionate), alkyl phosphate salts and ethoxylated derivatives thereof (e.g., potassium cetyl phosphate, oleath-10 phosphate, etc.). Examples of cationic emulsifiers include, but are not limited to, the following: behentrimonium chloride, distearyl dimethyl ammonium chloride, distearylammonium chloride, and combinations thereof. Examples of nonionic emulsifiers include, but are not limited to, the following: polyglycerol fatty acid esters (e.g., polyglyceryl-5 stearate, polyglyceryl-4 caprate, polyglyceryl-4 laurate, etc.), alkyl polyglycosides (e.g., cetearyl glucoside), ethoxylated fatty alcohols (e.g., ceteth-20, oleth-10, ceteareth-20, laureth-10, etc.) ethoxylated fatty acid esters or polyethylene glycol (PEG) ester derivatives (e.g., PEG-40 stearate), ethoxylated sorbitan fatty esters (e.g., polysorbate), ethoxylated glycerin monoesters (e.g., PEG-8 glyceryl laurate), ethoxylated castor oil derivatives (e.g., PEG-40 hydrogenated castor oil), ethoxylated derivatives of rapeseed oil (e.g., PEG-10 rapeseed sterol), ethoxylated lanolin, ethoxylated polyglyceryl ester (i.e., PEG-10 polyglyceryl-2 laurate), and combinations thereof. Examples of amphoteric emulsifiers include, but are not limited to, the following: betaine, a betaine derivative, an imidazoline derivative, an amino carboxylic acid, a protein, lecithin, and combinations thereof. Examples of betaine and betaine derivatives suitable for use as amphoteric emulsifiers include, but are not limited to, the following: alkyl betaines, amidoalkyl betaines, sulfobetaines, and acyl amino betaines, and combinations thereof.

Those of ordinary skill will appreciate that one or more emulsifying agents can be included in the cosmetic compositions disclosed herein, and that the emulsifying agent can be a mixture or combination of the different classes of emulsifiers (e.g., a mixture of a nonionic emulsifying agent and a cationic emulsifying agent). In one embodiment, the emulsifying agent comprises stearyl alcohol, cetearyl olivate, sorbitan olivate, and polyglyceryl-6-distearate.

7. Preservatives

Preservatives are substances that are used to inhibit the development of microorganisms, such as bacteria and mold, in a cosmetic product. Non-limiting examples of preservatives that can be used in the cosmetic compositions disclosed herein include, but are not limited to, the following: benzoic acid (and salts thereof including sodium benzoate), sorbic acid (and salts thereof, including potassium sorbate), salicylic acid, alcohols, organohalogen compounds, aldehydes, glycol ethers, parabens, and combinations thereof. Suitable organohalogen compounds include, but are not limited to, triclosan, methylisothiazolinone, methylchloroisothiazolinone, chlorphenesin, chloroxylenol, iodopropynyl butylcarbamate, methyldibromo glutaronitrile, and combinations thereof. Examples of suitable aldehydes include, but are not limited to, formaldehyde, benzylhemiformal, diazolidinyl urea, imidazolidinyl urea, 2-bromo-2-nitropropane-1,3-diol, dmdm hydantoin. mdm hydantoin, quaternium-15, sodium hydroxymethyl glycinate, and combinations thereof. Glycol ether suitable as preservatives in the cosmetic compositions disclosed herein include, but are not limited to, phenoxyethanol, 2-butoxyethanol, 2-(2-butoxyethoxy)-ethanol, 2-(2-ethoxy)-ethanol, and combinations thereof. Paraben suitable for use as a preservative include, but are not limited to, methylparaben, ethylparaben, propylparaben, butylparaben, isobutylparaben, and combinations thereof.

8. Chelating Agents

Chelating agents are ingredients that bind with metal ions and play a crucial role in the stability and efficacy of cosmetic compositions. Non-limiting examples of chelating agents include, but are not limited to, the following: EDTA (e.g., disodium EDTA and tetrasodium EDTA), GLDA (Glutamic acid diacetate), phytic acid, and combinations thereof. Other chelating agents suitable for use in the cosmetic compositions disclosed herein will be known to those of ordinary skill in the art.

9. Buffering Agents

In the cosmetic compositions disclosed herein, a buffering agent or a pH adjusting agent can be used to adjust the pH of the cosmetic composition to a pH ranging from about 4.0 to about 6.5. Non-limiting examples of buffering agents that can be used to adjust the pH of the cosmetic composition include, but are not limited to, acids, hydroxides, phosphates, alkalonamines, and combinations thereof. In one embodiment, the buffering or pH adjusting agent is citric acid.

10. Coloring Agents

Coloring agents are substances that color other substances in a cosmetic composition that are devoid of color (i.e., transparent, white, or gray). In some embodiments, the cosmetic compositions include a coloring agent. Non-limiting examples of coloring agents include, but are not limited to, dyes, pigments, colorants, or other substances that can impart color to the cosmetic composition.

11. Fragrances

In certain aspects of the present invention, the cosmetic composition is fragrance-free (i.e., no scent). In other aspects, however, the cosmetic composition can include one or more fragrances. Non-limiting examples of suitable fragrances include, but are not limited to, the following: perfumes, essential oils and aromatic compounds. In one embodiment, the fragrance is the fragrance is a combination of Litsea cubeba fruit oil and Cinnamomum camphora (Camphor) bark oil.

12. UV Filtering Agents

In certain aspects, the cosmetic compositions can include UV absorption and/or reflecting agents (e.g., chemical and physical sunblocks). In some embodiments, the cosmetic compositions include a UV filter agent that is either an organic filters and inorganic filters. Non-limiting examples of organic UV filter include, but are not limited to, the following: aminobenzoic acid, avobenzone, cinoxate, dioxybenzone, homosalate, meradimate, octocrylene, octinoxate, octisalate, oxybenzone, padimate O, ensulizole, sulisobenzone, trolamine salicylate, and combinations thereof. Non-limiting examples of organic UV filter include, but are not limited to, the following: zinc oxide and titanium oxide.

13. Amounts of the Fermented Grate Concentrate and Other Cometically Acceptable Ingredients

It is contemplated that the compositions of the present invention can include any amount of the ingredients discussed in this specification. The cosmetic compositions can also include any number of combinations of additional ingredients described throughout this specification, but will include the fermented grape concentrate in addition to at least one other cosmetically acceptable ingredient (e.g., an active agent, an emulsifying agent, a pigments, or additional cosmetically acceptable ingredients). Those of ordinary skill will appreciate that the concentrations of the fermented grape concentrate and any ingredient within the cosmetic compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4% 1.5%, 1.6%, 1.7%, 1.8%, 1.9%2.0% 2.1%, 2.2%, 2.3%, 2.4%, 2.5%, 2.6%, 2.7%, 2.8%, 2.9%, 3.0%, 3.1%, 3.2%, 3.3% 3.4%, 3.5%, 3.6%, 3.7%, 3.8%, 3.9%, 4.0%, 4.1%, 4.2%, 4.3% 4.4% 4.5% 4.6% 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4% 7.5% 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 1%, %16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of the fermented grape concentrated and/or at least one of the ingredients that are mentioned throughout the specification and claims. In non-limiting aspects, the percentage can be calculated by weight or volume of the total composition. For instance, in the table below, the percentages are based on weight. A person of ordinary skill in the art would understand that the concentrations can vary depending on the addition, substitution, and/or subtraction of ingredients in a given composition.

In some embodiments, the cosmetic compositions disclosed herein include the following components at the ranges:

| Component | Range |
|---|---|
| Cosmetically Acceptable Ingredient | (by wt.) |
| Fermented Grape Concentrate/Solvent | >30% |
| Humectant | 0.5%-20% |
| Emulsifying Agent | 0.1%-5% |
| Emollient | 0.1%-20% |
| Rheology-modifying Agent | 0.01%-2% |
| Active Agent(s) | <15% |
| Buffering Agent | <1% |
| Preservative | <5% |
| Fragrance | <2% |

In one embodiment, the solvent (including the fermented grape concentrate) is present in the cosmetic composition in an amount ranging from about 30% to about 100%, preferably from about 50% to about 98%, preferably, from about 75% to about 96%. In one embodiment, the humectant is present in the cosmetic composition in an amount ranging from about 0.5% to about 20%, preferably from about 3% to about 15%, preferably, from about 5% to about 10%. In one embodiment, the emulsifying agent is present in the cosmetic composition in an amount ranging from about 0.10% to about 20%, preferably from about 0.5% to about 10%, preferably, from about 1% to about 5%. In one embodiment, the rheology-modifying agent is present in the cosmetic composition in an amount ranging from about 0.01% to about 2%, preferably from about 0.02% to about 1%, preferably, from about 0.03% to about 0.10%. In one embodiment, the active agent is present in the cosmetic composition in an amount that is less than 15%, and preferably in an amount ranging from about 0.01% to about 2%, preferably from about 0.10% to about 2%, preferably, from about 0.04% to about 1%. In one embodiment, the buffering agent (or pH adjuster) is present in the cosmetic composition in a positive amount, but in an amount less than 1%, less than 0.5%, and less than 0.05%. In one embodiment, the preservative is present in the cosmetic composition in a positive amount, but in an amount less than 5%, less than 2%, less than 1%, less than 0.5%, and less than 0.05%. In one embodiment, the fragrance is present in the cosmetic composition in a positive amount, but in an amount less than 2%, less than 1%, less than 0.50%, and less than 0.10%.

### D. Methods of Use

In another aspect, the present invention provides a method of improving a condition or appearance of skin, the method comprising applying to the skin an effective amount of a topical cosmetic composition, the composition comprising: the cosmetic composition comprising: a fermented grape (Vitis vinifera) concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and a cosmetically acceptable ingredient; wherein the pH of the cosmetic composition ranges from about 4.0 to about 6.5. In one embodiment, the fermented grape concentrate has one or both of the following characteristics: (i) a sugar content of 3° Bx or less; and/or (ii) an alcohol by volume (ABV) of between about 0% and about 6%. In one embodiment, the topical composition is a moisturizer, such as a lotion, a cream, a gel, or a serum.

In one embodiment, by applying the topical cosmetic composition to the skin, the skin is hydrated. In one embodiment, by applying the topical cosmetic composition to the skin, the skin is treated to increase moisture level in the skin. In one embodiment, by applying the topical cosmetic composition to the skin, the skin radiance is increased. In one embodiment, by applying the topical cosmetic composition to the skin, the skin is treated to inhibit melanogenesis and/or tyrosinase. In one embodiment, by applying the topical cosmetic composition to the skin, the skin is treated to inhibit hyperpigmentation. In one embodiment, by applying the topical cosmetic composition to the skin, collagen production in the skin is increased. In one embodiment, by applying the topical cosmetic composition to the skin, the skin is treated to improve a skin condition, wherein the skin conditions is selected from skin radiance, skin tone clarity, skin brightness, skin tone evenness, and combinations thereof. In one embodiment, by applying the topical cosmetic composition to the skin, the skin is treated to reduce a skin condition, wherein the skin condition is photo damage, loss of facial firmness, fine lines, deep lines, wrinkles, skin dullness, skin sagging, appearance of age spots on skin, or combinations thereof.

It is also contemplated that the cosmetic compositions disclosed throughout this specification can be used as a leave-on or rinse-off composition. By way of example, a leave-on composition can be one that is topically applied to skin and remains on the skin for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the skin and then removed or rinsed from the skin (e.g., with water) within a period of time such as less than 5, 4, 3, 2, or 1 minute. An example of a rinse off composition can be a skin cleanser, shampoo, conditioner, or soap. An example of a leave-on composition can be a skin moisturizer, such as a serum, sunscreen, mask, overnight cream, or a day cream.

The compositions and methods of the present invention can be used on or in connection with any mammal, including both human and non-human mammals. In a preferred embodiment, the compositions and methods are used to improve a condition or appearance of skin of a human.

### E. Kits

Kits are also provided by the present invention. For instance, cosmetic compositions of the present invention can be included in a kit. A kit can include a container. Containers can include, for example, a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### F. Examples

1. Method for Preparing the Fermented Grape Concentrate

Sagrantino grapes, which are a red/purplish grape, were selected to be cultivated, harvested, and processed for their high antioxidant capacity juice for use in various cosmetic products. The Sagrantino grapes were grown on the Juice Beauty Farm in Dry Creek Valley (Healdsburg, Sonoma County, CA), where the soil is rich and the days are hot and nights are cold, all of which are perfect conditions for growing grapes. Additionally, the farm that was selected for growing the Sagrantino grapes is located on a hillside, which protects the grapes from freezing conditions that can at times ruin the grapes, especially when the grapes are grown on the adjacent valley floor). The Sagrantino grapes were cultivated, harvested, and then processed via fermentation and extraction using techniques and methodologies similar to those used in the wine industry to produce the fermented grape concentrate disclosed herein. The fermentation process generally takes anywhere from 14 to 21 days.

Cultivation: After the initial harvest and antioxidant testing, the goal was to gain a higher antioxidant value so the farming techniques of the Sagrantino grape were adjusted as follows: In April, irrigation and fertilization began and continued through harvest. From May to August, organic-approved sprays were used to control mildew and this spraying was carried out several times per month from May to August. In May and June, caning, which is a specialized form of pruning, was started to control the canopy and fruit yield. In July, the leafing started to enhance both ventilation and sunlight. The Sagrantino vineyard was leafed aggressively or intentionally “over-leafed” to enhance the antioxidant and ultraviolet absorption (SPF production) capacity of the grapes. The cultivation of the Sagrantino grapes was continued through August and into September.

Harvest: The Sagrantino grapes were harvested in September, which is earlier in the year than when harvesting typically occurs for other red grapes. The grapes were then crushed, fermented and processed to prepare the high antioxidant fermented grape concentrate using methods known to those of ordinary skill in the art and as exemplified by the methods described below. After the harvesting of the Sagrantino grapes, the grapevines were immediately fertilized and irrigated until they went formant for the winter. In the Winter, pruning occurred and cover crops were planted. The cover crop was changed from rye grasses to clover. All of the inputs used were from OMRI certified organic so all mildew and other agents were organic approved ingredients rather than conventional, non-organic ingredients. All fertilizers used were organic approved fertilizers. The watering, leafing, and pruning continued in the same way from harvest to harvest.

Processing—Fermentation and Extraction: A long maceration of the fruit was carried out during the fermentation process in order to maximize the concentration of antioxidants extracted during this period. 50% of phenolic compounds are present in the seeds of the Sagrantino grapes so fermentation period was longer in order to extract the phenolic compounds from the seeds. Maceration was carried out throughout the process, which means passively extracting in the tank by continuously ensuring that the skins were kept wet in the alcohol environment. This longer fermentation process would not be carried out with wines as it would likely turn the taste of the wine to more of a vinegar taste.

In essence, the grapes were pressed early and were crushed about 16 days after the press. The Sagrantino juice initially had a high brix. The grapes were treated in a manner similar to how grapes for a very high-end, fine wine would be treated (rather than a large distribution, mass wine). The Sagrantino grape juice was made first and then left to let the tannins naturally decline with the fermentation process.

Sugar was also reduced or removed through ethanolic fermentation to stabilize the juice and to enhance the shelf-life stability of the fermented grape concentrate. The fermentation process not only removed the sugar from the extract, preventing the product from becoming sticky and unstable, but it also led to an increase in alcohol content. The alcohol can increase the polyphenols extracted into solution, leading to an enhanced antioxidant level. Thereafter, a “clean” reverse osmosis membrane process was used to reduce or remove ethanol/alcohol, resulting in a fermented grape concentrate with a lower ABV, but with an enhanced stability due to the reduction in sugar content. Those of skill will appreciate that other methods, such as through the use of a spinning cone column or chemical treatment, could also be used to reduce or remove the alcohol. Naturally occurring malic acid was left in the final product because it has positive for skincare cosmetics, which is in contrast to wines were malic acid is removed through a separate malic acid removal step.

The fermented grape concentrate was processed until it had one or more of the following characteristics: (i) an Oxygen Radical Absorbance Capacity (ORAC) value ranging from about 10 μM TE/g to about 60 μM TE/g, (ii) a total phenolic content ranging from about 1000 mg/L to about 4500 mg/L; (iii) a sugar content of 3° Bx or less; and (iv) an alcohol by volume (ABV) of between about 0% and about 6%.

The final juice product was filtered and then frozen until used to prepare the desired cosmetic compositions/products.

Exemplary data for the 2019 and 2020 Sagrantino Harvests relative to the organic grape juice, which Juice Beauty purchases from various brokers in 2019 and which is a blend of red and white grape juices, is shown in the able below:

TABLE 1
ORAC and Total Phenolics

| | | Organic Juice | 2019 | 2020 | | |
|---|---|---|---|---|---|---|
| | | blend bought | Sagrantino | Sagrantino | Sagrantino | |
| | Method/Labs | in 2019 | Harvest | Harvest | Average | Variance |
| ORAC total (μM TE/g) | /ABC testing | 3.68 | 27.03 | 31.26 | 29.145 | 4.473225 |
| ORAC - hydro | /ABC testing | 3.26 | 23.91 | 28 | 25.955 | 4.182025 |
| ORAC - lipc | /ABC testing | 0.42 | 3.12 | 3.26 | 3.19 | 0.0049 |
| Total phenolics (FOUN) (mg/L) | SOP3.6.38/ABC testing | 500 | 2000 | 3400 | 2700 | 490000 |
| Catechin (mg/L) | HPLC/Enartis | | 121 | | | |
| Monomeric Flavan-3-ols (mg/L) | HPLC/Enartis | | 858 | | | |
| Polymeric Flevan-3-ols (mg/L) | HPLC/Enartis | | 476 | | | |
| Phenolic acids (mg/L) | HPLC/Enartis | | 30 | | | |
| Hydrocinnamates (mg/L) | HPLC/Enartis | | 166 | | | |
| Flavonols (mg/L) | HPLC/Enartis | | 31 | | | |
| Monomeric anthocyenins (mg/L) | HPLC/Enartis | | 220 | | | |
| Polymeric anthocyanins (mg/L) | HPLC/Enartis | | 11 | | | |
| Quercetin (mg/L) | HPLC/Enartis | | <1 | | | |
| Quercetin glucosides (mg/L) | HPLC/Enartis | | 27 | | | |
| Cetechin/Tannin Ratio | HPLC/Enartis | | 0.25 | | | |
| Poly Anthocyanins/Tannin Ratio | HPLC/Enartis | | 0.02 | | | |
| Total color @524 nm | FCCX8/ABS testing | | | 6.67 | | |

As shown in Table 1, the ORAC values for the 2019 and 2020 Sagrantino Harvests are orders of magnitude higher than the ORAC value for Smart Fruit Harvest, which contains a higher antioxidant level compared to most other grape juice that have a lower phenolic content due to the processing used to prepare the juice. It is surprisingly and unexpectedly been discovered that the 2020 Sagrantino Harvest has an ORAC value that is over seven (7) times higher than the Smart Fruit Harvest, and that the 2019 Sagrantino Harvest has an ORAC value that is over four (4) times higher than the Smart Fruit Harvest. As also shown in Table 1, the total phenolic content for the 2019 and 2020 Sagrantino Harvests are orders of magnitude higher than the total phenolic content of Smart Fruit harvest. It has been surprisingly discovered that crushing the grapes, rather than squeezing and concentrating the juice, keeps the skins while removing some of the sugar, and keeping the skins helps to increase the phenolic content and, in turn, the antioxidant levels of the resulting product.

2. Preparation of the Stem Cellular SuperGrape Youth Renew Serum

A serum according to the present invention was prepared using the following ingredients in descending order by volume:

aloe barbadensis leaf juice*, vitis vinifera (Sagrantino grape) juice†, pyrus malus (apple)
juice*, pentylene glycol†, glycerin*, stearyl alcohol†, simmondsia chinensis (jojoba) seed oil*,
malus domestica fruit cell culture extract†, vitis vinifera (grape) extract†, opuntia ficus-indica
stem extract†, hyaluronic acid, panthenol (Vitamin B5), ascorbyl glucoside (Vitamin C),
tocopherol (Vitamin E)†, butyrospermum parkii (shea) butter*, hydrolyzed jojoba esters†,
algin*, borago officinalis seed oil*, rosa canina fruit oil*, linum usitatissimum (linseed) seed
oil*, arginine, cetearyl olivatet, sorbitan olivate†, polyglyceryl-6 distearate†, xanthan gum,
citric acid, potassium sorbate, sodium benzoate, litsea cubeba fruit oil†, cinnamomum
camphora (camphor) bark oil†.
*Certified Organic Ingredient
†Plant Ingredient

In one embodiment, the above list of ingredients, which includes the fermented grape concentrate referred to as “Vitis vinifera (Sagrantino grape) juice†,” was combined in the following amounts to prepare a cosmetic composition in the form of a serum that is both high in phenolic content and high in antioxidants:

| FUNCTION | INGREDIENT | Range |
|---|---|---|
| Solvent | FERMENTED GRAPE CONCENTRATE, ALOE BARBADENSIS LEAF JUICE, | >30% |
| | PYRUS MALUS (APPLE) JUICE | |
| Humectant | PENTYLENE GLYCOL, GLYCERIN | 0.5%-20% |
| Emulsifying Agent | STEARYL ALCOHOL, CETEARYL OLIVATE, SORBITAN OLIVATE, POLYGLYCERYL-6 DISTEARATE | 0.1%-5% |
| Emollient | SIMMONDSIA CHINENSIS (JOJOBA) SEED OIL, BORAGO OFFICINALIS SEED OIL, ROSA CANINA | 0.1%-20% |
| | FRUIT OIL, LINUM LISITATISSIMUM (LINSEED) SEED OIL, BUTYROSPERMUM PARKII (SHEA) | |
| | BUTTER*, HYDROLYZED JOJOBA ESTERS | |
| Rheology Modifier | ALGIN, XANTHAN GUM | 0.01%-2% |
| Actives | OPUNTIA FICUS-INDICA STEM EXTRACT, MALUS DOMESTICA FRUIT CELL CULTURE EXTRACT, | <15% |
| | VITIS VINIFERA (GRAPE) EXTRACT, TOCOPHEROL, PANTHENOL, HYALURONIC ACID, ASCORBYL | |
| | GLUCOSIDE, ARGININE | |
| Buffering agent | CITRIC ACID | <1% |
| Preservative | POTASSIUM SORBATE, SODIUM BENZOATE | <5% |
| Fragrance | LITSEA CUBEBA FRUIT OIL, CINNAMOMUM CAMPHORA (CAMPHOR) BARK OIL | <2% |

The serum was mixed and prepared at a licensed, professional cosmetics lab by a Ph.D. scientist using generally accepted cosmetics protocols. The lab is GMP (Good Manufacturing Process) Certified,

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A topical cosmetic composition, the cosmetic composition comprising:
a fermented grape (Vitis vinifera) concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and
a cosmetically acceptable ingredient;
wherein the pH of the cosmetic composition ranges from about 4.0 to about 6.5.

2. The cosmetic composition of claim 1, wherein the grape concentrate has an ORAC value ranging from about 15 μM TE/g to about 50 μM TE/g.

3. The cosmetic composition of claim 2, wherein the grape concentrate has an ORAC value ranging from about 20 μM TE/g to about 40 μM TE/g.

4. The cosmetic composition of claim 1, wherein the grape concentrate has a total phenolic content ranging from about 1500 mg/L to about 3600 mg/L.

5. The cosmetic composition of claim 1, wherein the grape concentrate is a Sagrantino grape concentrate.

6. The cosmetic composition of claim 1, wherein the pH of the cosmetic composition ranges from about 4.8 to about 5.8.

7. The cosmetic composition of claim 1, wherein the cosmetically acceptable ingredient is selected from the group consisting of an active agent, a solvent, a humectant, a rheology modifying agent, an emollient, an emulsifying agent, a preservative, a chelating agent, a buffering agent, a colorant, a fragrance, a UV filtering agent, and combinations thereof.

8. The cosmetic composition of claim 1, wherein the cosmetically acceptable ingredient is a combination of an agent, a solvent, a humectant, a rheology modifying agent, and a preservative.

9. The cosmetic composition of claim 8, further comprising one or more cosmetically acceptable ingredients selected from the group consisting of an emollient, an emulsifying agent, a buffering agent, a colorant, a fragrance, and a UV filtering agent.

10. The cosmetic composition of claim 8, wherein the solvent is selected from the group consisting of water, a plant juice, a plant water, and combinations thereof.

11. The cosmetic composition of claim 10, wherein the plant juice is selected from the group consisting of Aloe arborescens leaf juice, Aloe barbadensis leaf juice, Ananas sativus (pineapple) fruit juice, Betula alba (birch) juice, Borojoa patinoi fruit juice, Bulbine frutescens leaf juice, Citrus aurantium dulcis (Orange) fruit extract, Citrus medica limonum (Lemon) juice, Cocos nucifera (Coconut) fruit juice, Cucumis sativus (Cucumber) juice, Empetrum Nigrum (black Crowberry) Fruit Juice, Fragaria chiloensis (Strawberry) fruit juice, Hippophae rhamnoides fruit juice, Hordeum vulgare leaf juice, Musa paradisiaca fruit juice, Polymnia sonchifolia root juice, Prunus persica (Peach) juice, Pyrus malus (Apple) juice, Rubus chamaemorus (Cloudberry) fruit juice extract, Sorghum bicolor stalk juice, Vaccinium myrtillus (Bilberry) fruit juice, Vitis vinifera (Grape) juice, and combinations thereof.

12. The cosmetic composition of claim 10, wherein the plant water is selected from the group consisting of Actinidia Chinensis (Kiwi) fruit water, Ananas sativus (Pineapple) fruit water, Anthemis nobilis flower water, Citrullus lanatus (Watermelon) fruit extract, Citrullus vulgaris (Watermelon) fruit extract, Citrus aurantium dulcis (Orange) fruit water, Citrus grandis (Grapefruit) fruit water, Citrus paradisi (Grapefruit) fruit water, Citrus medica limonum (Lemon) fruit water, Ficus carica (Fig) fruit water, Fragaria ananassa (Strawberry) fruit water, Gossypium herbaceum (Cotton) fruit water, Malus domestica (apple) fruit water, Mentha piperita leaf water, Olea europaea (Olive) fruit water, Opuntia ficus-indica (prickly pear) water, Prunus armeniaca (plum) fruit water, Prunus persica (plum) fruit water, Prunus armeniaca (Apricot) fruit water, Prunus cerasus (Cherry) fruit water, Prunus persica (Peach) fruit water, Rubus fruticosus fruit (blackberry) water, Rubus idaeus (Raspberry) fruit water, Vaccinium macrocarpon (Cranberry) fruit water, Vaccinium myrtillus (bilberry) fruit water, Vanilla planifolia fruit water, Vitis vinfera (Grape) fruit water, and combinations thereof.

13. The cosmetic composition of claim 10, wherein the solvent comprises Aloe barbadensis leaf juice and Pyrus malus (apple) juice.

14. The cosmetic composition of claim 8, wherein the humectant is selected from the group consisting of a glycol, a glycerin, a diglycerin and combinations thereof.

15. The cosmetic composition of claim 9, wherein the humectant is a glycol and the glycol is selected from the group consisting of butylene glycol, pentylene glycol, propanediol, polyethylene glycol (PPG), and combinations thereof.

16. The cosmetic composition of claim 9, wherein the humectant comprises pentylene glycol and glycerin.

17. The cosmetic composition of claim 8, wherein the rheology modifying agent is selected from the group consisting of a gum, carrageenan, a carrageenan derivative thereof, a carbomer, a carbomer derivative and thereof, acrylic acid, an acrylic acid derivative, cellulose, a cellulose derivative, a silica, a silica derivative, clay, dextrin, a dextrin derivative, a copolymer, and combinations thereof.

18. The cosmetic composition of claim 17, wherein the rheology modifying agent is a gum and the gum is selected from the group consisting of xanthan gum, algin, sclerotium gum, dehydroxanthan gum, Ceratonia siliqua gum, Caesalpinia espinosa gum, cellulose gum, and combinations thereof.

19. The cosmetic composition of claim 17, wherein the rheology modifying agent is carrageenan or a carrageenan derivative selected from the group consisting of chrondrus crispus powder, sodium carrageenan, and combinations thereof.

20. The cosmetic composition of claim 17, wherein the rheology modifying agent is a carbomer or a carbomer derivative, wherein the carbomer derivative is sodium carbomer.

21. The cosmetic composition of claim 17, wherein the rheology modifying agent is acrylic acid or an acrylic acid derivative selected from the group consisting of acrylates copolymer, sodium acrylates copolymer, acrylates/C10-30 alkyl acrylates crosspolymer, polyacrylate crosspolymer-6, polyquaternium-37, sodium polyacrylate, and combinations thereof.

22. The cosmetic composition of claim 17, wherein the rheology modifying agent is cellulose or a cellulose derivative selected from the group consisting of cellulose acetate, cellulose gum, ethyl cellulose, and combinations thereof.

23. The cosmetic composition of claim 17, wherein the rheology modifying agent is a silice derivates selected from the group consisting of calcium silicate, silica, magnesium silicate, and combination thereof.

24. The cosmetic composition of claim 17, wherein the rheology modifying agent is a clay, wherein the clay is hectorite or bentonite.

25. The cosmetic composition of claim 17, wherein the rheology modifying agent is dextrin or a dextrin derivative.

26. The cosmetic composition of claim 17, wherein the rheology modifying agent comprises algin and xanthan gum.

27. The cosmetic composition of claim 8, wherein the preservative is selected from the group consisting of benzoic acid, sorbic acid, salicylic acid, alcohols, organohalogen compounds, aldehydes, glycol ethers, parabens, and combinations thereof.

28. The cosmetic composition of claim 27, wherein the preservative comprises potassium sorbate and sodium benzoate.

29. The cosmetic composition of claim 8, wherein the active agent is selected from the group consisting of a vitamin, a vitamin derivative, an amino acid, an amino acid derivative, a peptide, a peptide derivative, a protein, a protein derivative, a polysaccharide; a plant extract; an alpha-, beta-, or poly-hydroxy acid (AHA); urea; uric acid derivatives; and combinations thereof.

30. The cosmetic composition of claim 29, wherein the active agent is a plant extract selected from the group consisting of Opuntia ficus-indica stem extract, Malus domestica fruit cell culture extract, and Vitis vinifera (grape) extract.

31. The cosmetic composition of claim 29, wherein the active agent is a vitamin or vitamin derivative selected from the group consisting of tocopherol (Vitamin E), ascorbic acid (Vitamin C), panthenol, retinol (Vitamin A), niacinamide, and combinations thereof.

32. The cosmetic composition of claim 29, wherein the active agent is a polysaccharide, wherein the polysaccharide is hyaluronic acid.

33. The cosmetic composition of claim 29, wherein the active agent is an amino acid selected from the group consisting of arginine, histidine, methionine, lysine, proline, leucine, glycine, and combinations thereof.

34. The cosmetic composition of claim 29, wherein the active agent is a peptide selected from the group consisting of acetyl hexapeptide-8, dipeptide-2, hexapeptide-11, hydrolyzed wheat peptides, oligopeptide-1.

35. The cosmetic composition of claim 29, wherein the active agent is a protein selected from the group consisting of collagen, elastine, keratin, and combinations thereof.

36. The cosmetic composition of claim 29, wherein the active agent is an alpha-, beta-, or poly-hydroxy acid selected from the group consisting of lactic acid, glycolic acid, malic acid, tartaric acid, salicylic acid, gluconolactone, and combinations thereof

37. The cosmetic composition of claim 29, wherein the active agent comprises a uric acid derivative, wherein the uric acid derivative is allantoin.

38. The cosmetic composition of claim 29, wherein the active agent comprises urea.

39. The cosmetic composition of claim 33, wherein the active agent comprises Opuntia ficus-indica stem extract, Malus domestica fruit cell culture extract, and Vitis vinifera (grape) extract, tocopherol, panthenol, hyaluronic acid, ascorbyl glucoside, and arginine.

40. The cosmetic composition of claim 8, further comprising an emollient, wherein the emollient is selected from the group consisting of a triglyceride, an oil, a butter, a wax, a silicone, mineral oil, a paraffin hydrocarbon, a fatty alcohol, a fatty acid, an ester of a fatty acid with an alcohol, a ceramide, a phospholipid, and combinations thereof.

41. The cosmetic composition of claim 40, wherein the emollient is selected from the group consisting of Simmondsia chinensis (Jojoba) seed oil, Borago officinalis seed oil, Rosa canina fruit oil, Linum usitatissimum (Linseed) seed oil, Butyrospermum parkii (Shea) butter, hydrolyzed jojoba esters, and combinations thereof.

42. The cosmetic composition of claim 8, further comprising an emulsifying agent, wherein the emulsifying agent is selected from the group consisting of anionic emulsifiers, cationic emulsifiers, non-ionic emulsifiers, amphoteric emulsifiers, and combinations thereof.

43. The cosmetic composition of claim 8, wherein the emulsifying agent is an anionic emulsifier selected from the group consisting of a fatty alcohol sulphate, an isethionate, an alkyl phosphate salt and ethoxylated derivatives thereof.

44. The cosmetic composition of claim 8, wherein the emulsifying agent is a cationic emulsifier selected from the group consisting of behentrimonium chloride, distearyl dimethyl ammonium chloride, distearylammonium chloride, and combinations thereof.

45. The cosmetic composition of claim 8, wherein the emulsifying agent is a non-ionic emulsifier selected from the group consisting of polyglycerol fatty acid esters, alkyl polyglycosides, ethoxylated fatty alcohols, ethoxylated fatty acid esters or polyethylene glycol (PEG) ester derivatives, ethoxylated sorbitan fatty esters, ethoxylated glycerin monoesters, ethoxylated castor oil derivatives, ethoxylated derivatives of rapeseed oil, ethoxylated lanolin, ethoxylated polyglyceryl ester, and combinations thereof.

46. The cosmetic composition of claim 8, wherein the emulsifying agent is an amphoteric emulsifier selected from the group consisting of betaine, a betaine derivative, an imidazoline derivative, an amino carboxylic acid, a protein, lecithin, and combinations thereof.

47. The cosmetic composition of claim 43, wherein the emulsifying agent comprises stearyl alcohol, cetearyl olivate, sorbitan olivate, and polyglyceryl-6-distearate.

48. The cosmetic composition of claim 8, further comprising a buffering agent, wherein the buffering agent is selected from the group consisting of acids, hydroxides, phosphates, alkalonamines, and combinations thereof.

49. The cosmetic composition of claim 48, wherein the buffering agent is citric acid.

50. The cosmetic composition of claim 8, further comprising a chelating agent.

51. The cosmetic composition of claim 52, wherein the chelating agent is selected from the group consisting of EDTA, GLDA, phytic acid, and combinations thereof.

52. The cosmetic composition of claim 8, further comprising a UV filter.

53. The cosmetic composition of claim 52, wherein the UV filter is selected from the group consisting of organic filters, inorganic filters, and combinations thereof.

54. The cosmetic composition of claim 8, further comprising a fragrance, wherein the fragrance is selected from the group consisting of perfumes, essential oils, aromatic compounds, and combinations thereof.

55. The cosmetic composition of claim 54, wherein the fragrance is selected from the group consisting of Litsea cubeba fruit oil, Cinnamomum camphora (Camphor) bark oil.

56. The cosmetic composition of claim 1, wherein the cosmetic composition is selected from the group consisting of a moisturizer, a mask, a body care product, an eye care product, a primer, a foundation, a concealer, a cream blush, a lipstick, a lip balm, a mascara, a shampoo, a hair conditioner, a shower gel and a facial cleanser.

57. The cosmetic composition of claim 56, wherein the cosmetic composition is a moisturizer, wherein the moisturizer is a lotion, a cream, a gel, or a serum.

58. A method of improving a condition or appearance of skin, the method comprising applying to the skin an effective amount of the topical cosmetic composition of any one of claims 1-57.

59. The method of claim 58, wherein the topical cosmetic composition is a serum.

60. The method of claim 58, wherein by applying the topical cosmetic composition, the skin is hydrated.

61. The method of claim 58, wherein by applying the topical cosmetic composition, the skin radiance is increased.

62. The method of claim 58, wherein by applying the topical cosmetic composition, the skin is treated to inhibit melanogenesis and/or tyrosinase.

63. The method of claim 58, wherein by applying the topical cosmetic composition, the skin is treated to inhibit hyperpigmentation.

64. The method of claim 58, wherein by applying the topical cosmetic composition, collagen production in the skin is increased.

65. The method of claim 58, wherein by applying the topical cosmetic composition, the skin is treated to improve a skin condition, wherein the skin conditions is selected from skin radiance, skin tone clarity, skin brightness, skin tone evenness, and combinations thereof.

66. The method of claim 58, wherein by applying the topical cosmetic composition, the skin is treated to reduce a skin condition, wherein the skin condition is photo damage, loss of facial firmness, fine lines, deep lines, wrinkles, skin dullness, skin sagging, appearance of age spots on skin, or combinations thereof

67. The method of claim 58, wherein by applying the topical cosmetic composition, the skin is treated to increase moisture level in the skin.

68. A topical cosmetic composition, the cosmetic composition comprising:
a fermented grape concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and
a cosmetically acceptable ingredient, wherein the cosmetically acceptable ingredient comprises an active agent; a solvent, a humectant, a rheology modifying agent, and a preservative;
wherein the pH of the cosmetic composition ranges from about 4.0 to about 6.0.

69. A topical cosmetic composition, the cosmetic composition comprising:
a fermented grape concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L; and
a cosmetically acceptable ingredient, wherein the cosmetically acceptable ingredient comprises an active agent, a solvent, a humectant, an emulsifying agent, an emollient, a rheology modifying agent, a buffering agent, a preservative, and a fragrance;
wherein the pH of the cosmetic composition ranges from about 4.0 to about 6.5.

70. A method for preparing a topical cosmetic composition, the method comprising: (a) combining (i) a fermented grape concentrate, wherein the grape concentrate has an Oxygen Radical Absorbance Capacity (ORAC) value ranging from about 10 μM TE/g to about 50 μM TE/g, and a total phenolic content ranging from about 1000 mg/L to about 4500 mg/L, and (ii) a cosmetically acceptable ingredient to form a product; and (b) adjusting the pH of the product to a pH of about 4.0 to about 6.5, thereby preparing the topical cosmetic composition.

71. A composition for use in a topical cosmetic product, the composition comprising a fermented grape concentrate, wherein the grape concentrate has an Oxygen Radical Absorbance Capacity (ORAC) value ranging from about 10 μM TE/g to about 60 μM TE/g, and a total phenolic content ranging from about 1000 mg/L to about 4500 mg/L, and wherein the grape concentrate is stable at a pH of about 4.0 to about 6.5.

72. The cosmetic composition of any one of claims 1-57, wherein the grape concentrate has an alcohol by volume of between 0% and 6%.

73. The cosmetic composition of any one of claims 1-57, wherein the grape concentrate has a sugar content of 3° Bx or less.
